(19) United States
(12) Patent Application Publication
(10) Pub. No.: US 2026/0108247 A1
(43) Pub. Date: Apr. 23, 2026

(54) NERVE CONDUIT

(71) Applicant: Tissium S.A., Paris (FR)

(72) Inventors: Marie Malafosse, Paris (FR); Estelle Collin, Alfortville (FR); Elise Devries, San Francisco, CA (US); Joanna Xylas, Somerville, MA (US)

(21) Appl. No.: 19/120,487

(22) PCT Filed: Oct. 11, 2023
(86) PCT No.: PCT/EP2023/078267

§ 371 Date: Apr. 11, 2025

(30) Foreign Application Priority Data
Oct. 12, 2022 (WO) PCT/EP2022/078420

(51) Int. Cl. A61B 17/11; A61B 17/00

(52) U.S. Cl. CPC A61B 17/1128; A61B 2017/00004; A61B 2017/1132

(57) ABSTRACT

A nerve conduit for supporting repair of a nerve lesion and motor control and/or reducing risk of the development of a neuroma at the lesioned nerve end. The nerve conduit includes an elongate body formed by a wall and including a central portion defining an inner cavity and end portions defining a respective opening to the inner cavity and arranged adjacent to the central portion and at longitudinally opposing ends of the elongate body. One end portion is configured as an insertion portion configured for inserting a respective nerve end. The other end portion is configured as a nerve end outlet portion, an outer diameter of the wall defining the outlet portion is larger than an outer diameter of the wall defining the central portion.

### CROSS REFERENCE TO RELATED APPLICATIONS

This application is the U.S. National Stage of PCT/EP2023/078267 filed Oct. 11, 2023, which claims priority to PCT/EP2022/078420 filed Oct. 12, 2022, the entire contents of which are incorporated herein by reference in their entirety.

### TECHNICAL FIELD

The present disclosure relates to a nerve conduit for connecting a lesioned nerve end to a target tissue. In particular, the present disclosure relates to nerve conduits supporting repair of a nerve lesion and motor control or sensory function (e.g. skin sensitivity), and/or reducing risk of the development of a neuroma at the lesioned nerve end.

### BACKGROUND

Upon injury of a person, tissue damage may involve one or more nerve lesions of the peripheral nerve system resulting in a partial sensory loss and/or impaired motor skills. Such injuries involving nerve damage particularly occur in the lower limb and upper extremities, such as a hand or finger of a person, such that a person may experience a loss e.g. in tactile or haptic feedback and/or may have difficulties in controlling fine motor skills in the injured region, if the nerve lesion is not treated properly.

Current treatments of nerve lesions include coaptation of the nerve ends by various suturing techniques so as to provide a connection between the respective nerve ends in a substantially tensionless manner. In case a more severe defect is present, wherein the nerve ends are not directly adjacent to each other, a reconstruction may be required to overcome a corresponding gap. A reconstruction may be provided e.g. by an autologous or allogenic nerve graft. Alternatively, a reconstruction may be performed by providing a tubular structure so as to provide a nerve guide in the form of an inner lumen providing a directional path during neurogenesis. Such tubular structure, which accommodates the respective nerve ends, may be provided e.g. by autologous or allogenic venous structures or by artificially manufactured nerve conduits made of a biocompatible material. The use of a tubular structure may furthermore facilitate the repair of the lesion irrespective of the presence of a gap, e.g., by providing further mechanical support and structural stability, providing a tensionless repair, reducing an inflammatory response to the site of the lesion, and/or limiting the extent of fibrous tissue development.

However, in a clinical situation wherein the distal nerve end is lost, e.g. due to amputation or tissue removal, such connection between nerve ends is not possible. This not only complicates the treatment for at least partially restoring motor control or sensory function, but may also result in an increased risk of a neuroma, which may develop at the terminal end of a sectioned nerve. Such neuromas may develop from disorganized axons growing beyond the severed epineural sheath at the end of a transected nerve in an attempt to reinnervate distal muscle or skin, e.g. following severe trauma or surgery, wherein a sprouting of axons may form a bundle of disorganised and hyperexcitable nerve tissue (called neuroma) that does not have the original nerve structure. Neuromas are typically associated with neuropathic pain, numbness and tingling and may also interfere with the rehabilitation, the functional recovery and a sensory deficit. Treatments or attempts in prevention of neuromas may be based on suturing the proximal end of a lesioned nerve to a distinct motor nerve of an intact newly denervated muscle. The suturing technique may at least partially restore muscle control while reducing the risk of neuroma development.

Other surgical alternatives have been proposed to repair peripheral nerve injuries where the proximal nerve stump is unavailable or a significant nerve gap exists, or when nerve is transected too far from target organ or muscle or skin. For example, the End-to-side (ETS) nerve repair in which the distal stump of a damaged nerve is coapted to the side of an uninjured nerve (Lykissas, 2011, World Journal of Orthopaedics, vol. 2, no. 11, 102-106).

Another procedure called Targeted muscle reinnervation (TMR) is a surgical nerve-transfer procedure. According to TMR, residual nerves from the amputated limb are transferred to new muscle targets. As part of the nerve transfer, the target muscles are separated from their native motor nerve input so that the newly transferred nerve can reinnervate them (Bergmeister et al., 2021, Hand Clin, 37(3):415-424; Janes et al., 2021, Hand Clin, 37(3):345-359).

### SUMMARY

Starting from the known prior art there is a need to further facilitate the repair of nerve lesions, more particularly after amputation, resulting in nerve endings that can form neuromas or cause phantom limb pain.

According to the present disclosure it has been recognized that the connection of a proximal or distal nerve end of a nerve, in some embodiments a lesioned nerve, to target muscle tissue or target organ or side of target nerve may be difficult and/or may not provide a satisfying result, for example to restore (partial) motor control and/or sensory function and/or avoid the development of a neuroma and/or to result in regeneration of the injured nerve. Suturing the nerve end may result in undesired tissue damage and/or may complicate appropriate orientation of the terminal end of the proximal nerve end, such that the intended restoration of motor control or nerve coaptation is impaired. Furthermore, in the case of connection to target muscle tissue, suture technique into muscle tissue may complicate efficient securing of the nerve end. Alternative fixation methods using e.g. the application of a medical adhesive have been found to have limited success rates due to the small surface available for the application. Required curing times furthermore may result in a loss of proper positioning of the respective nerve end and/or an overall loss of proper attachment.

It is hence an object of the present disclosure to further facilitate the repair of nerve lesions and, in particular, to facilitate proper securing and positioning of nerve ends to a target tissue (e.g. muscle, bone or skin) or target organ or side of target nerve (collectively named target site). It may be a particular object of the present disclosure to facilitate the connection of a proximal nerve end to a target muscle or skin in an effective and efficient manner for the restoration of motor control and/or restoration of sensory feedback and/or preventing of neuroma development. It may be another object of the present disclosure to facilitate the connection of a proximal nerve end to the side of target (uninjured) donor nerve in effective and efficient End-to-side (ETS) nerve repair method. It may be another object of the present disclosure to facilitate the connection of nerve ends to organs, for example transplanted organs (e.g. heart, liver and kidney).

Said object is achieved by the claims. Embodiments are depicted in the claims, the description, and the Figures.

Accordingly, a nerve conduit for connecting a nerve end, e.g. a lesioned nerve end, to a target site (e.g. target tissue or target organ or side of target nerve) is suggested, comprising an elongate body formed by a wall, wherein the elongate body comprises a central portion defining an inner cavity and end portions defining a respective opening to the inner cavity and arranged adjacent to the central portion and at longitudinally opposing ends of the elongate body. One end portion is configured as a nerve end insertion portion configured for inserting a respective nerve end. According to the present disclosure, the other end portion is configured as a nerve end outlet portion, wherein an outer diameter of the wall defining the outlet portion is larger than an outer diameter of the wall defining the central portion.

By the increased diameter, connection to the target site is facilitated due to the accordingly enlarged surface area. This may not only increase the potential securing force between the outlet portion and the target site, but also facilitates correct application e.g. of a medical adhesive by indicating and/or delimiting a more prominent target application surface for the medical adhesive. The enlarged diameter of the outlet portion furthermore renders it easier to properly position and orientate the nerve conduit and the nerve end accommodated therein with regard to the target site, e.g. a denervated muscle or muscle section (e.g. muscle flap), a nerve side, bone or an organ, while providing increased stability upon placement of the nerve conduit. The outlet portion and/or the material thereof may furthermore be resilient so as to provide a level of adaptability to the anatomy of the target site. According to some embodiments, “target site” according to the present disclosure usually means a tissue, e.g. muscle, denervated muscle or muscle section (e.g. muscle flap) or bone, a nerve side or an organ.

The increased diameter of the outlet portion may further facilitate identification of the respective end portions, such that the surgeon readily understands which end portion is to be used for insertion of the nerve end to accommodate the nerve end within the insertion portion and inner cavity. In this regard, the nerve end of the respective nerve lesion may be secured to the nerve conduit after proper insertion of the nerve end into the corresponding insertion portion, wherein it may be ensured that a medical adhesive or other techniques used for securing the respective nerve end to the nerve conduit has been properly applied. The nerve end may hence be fixed or attached to the nerve conduit prior to connecting the nerve conduit to the target site.

The provision of the inner cavity and the enlarged outlet portion furthermore has the aspect that the nerve end may be observed or inspected after insertion into the insertion portion and the inner cavity. In particular, the respective nerve end may be received in the inner cavity via the corresponding insertion portion and may extend through the inner cavity towards or up to the corresponding outlet portion. In other words, the nerve end may be inserted in such manner that the nerve end terminates at or within the outlet portion or at the junction between the opening 20′ and the inner cavity, such that the nerve end may be observed via the outlet portion. Accordingly, proper insertion of the nerve end may be monitored prior to connection to the target site, and possible application of a medical adhesive.

The nerve conduit may generally comprise a tubular shape, wherein the wall defines an inner lumen or inner cavity with opposing openings so as to provide a continuous channel or through-hole. In other words, the wall forms a longitudinally extending elongated body, wherein a (fluid) connection or communication between the exterior and the inner cavity is provided via the respective openings of the end portions prior to the insertion of the nerve end and the connection of the nerve conduit to the target site.

In some embodiments, the cross-section of the inner cavity and openings is of circular or ellipsoid shape. A tubular or cylindrical shape may provide sufficient structural stability and may prevent sharp bends or kinking during tissue movement, i.e. compression or extension. The tubular shape may accordingly also provide that a homogeneous structure is provided, which reacts in a predefined manner along the entire central portion when forces act upon the central portion, e.g. upon impact.

In some embodiments, the maximum outer diameter of the wall defining the outlet portion is from 1 mm to 10 mm, from 2 mm to 6 mm, larger than the outer diameter of the wall defining the central portion. By providing the larger diameter by predefined dimensions, a predefined contacting surface or retention surface may be provided for the connection to the target site. The radial extension of the outlet portion may be dependent on the diameter of the nerve end to be accommodated in the nerve conduit and the corresponding diameter of the inner cavity. For example, nerve conduits having an inner cavity of from 1.5 mm to 4 mm may have an outlet portion having a diameter being e.g. 2 or 3 mm larger than the outer diameter of the wall defining central portion whereas nerve conduits having an inner cavity of about 8 mm to 10 mm may require a larger radial extension of the outlet portion to ensure proper and stabile connection to the target tissue, e.g. in the range of 6 mm to 10 mm larger than the outer diameter of the wall defining central portion. The predefined dimensions furthermore provide a corresponding surface e.g. for the application of a medical adhesive, such that a predefined fixation force or securing force may be ensured.

By the same token, a ratio between the maximum outer diameter of the wall defining the outlet portion and the outer diameter of the wall defining the central portion may be from 1.1:1.0 to 8.0:1.0, from 1.3:1.0 to 5.5:1.0, or from 1.1:1.0 to 3.0:1.0, or from 1.4:1.0 to 2,0:1,0. Accordingly, an enlarged outlet portion is provided that is readily identifiable and which facilitates the connection to the target site. The larger dimensions of the outlet portion may furthermore ensure that axonal growth may be directed to the target site even in the case of unintended misalignment or placement of the nerve conduit. For larger nerve ends with diameters exceeding e.g. 6 mm, absolute dimensions may limit the corresponding ratio in order to avoid potential incompatibility of the nerve conduit dimensions with the target site and surrounding tissue and/or the surgical procedure.

To accommodate a corresponding diameter of the nerve end, in some embodiments, the diameter of the inner cavity is from 1 mm to 12 mm, from 1.5 mm to 6.5 mm. Thereby, the nerve conduit is particularly desirable to accommodate nerve ends e.g. in the upper or lower extremities of a patient and for restoration of fine motor control and/or restoration of sensory feedback and/or preventing of neuroma development.

In some embodiments, in order to increase the structural stability of the connecting surface and limit the overall dimensions of the nerve conduit, a ratio between the longitudinal extension of the outlet portion and of the central portion is from 0.1:1.0 to 0.6:1.0. Such shorter longitudinal extension compared with the inner cavity may result in an outlet portion being formed as a radial flange, which facilitates proper orientation of the nerve conduit e.g. during the application of a medical adhesive and may provide an improved retention and/or securing surface for a medical adhesive or other attachment techniques.

In some embodiments, the radial extension of the outlet portion exceeds the longitudinal extension of the central portion, since the nerve end may be directly connected to the target site. In other words, the longitudinal extension of the central portion as a nerve guide may be dimensioned smaller, since a bridging of large gaps between the respective nerve end and the target site is not required.

Accordingly, in some embodiments, a ratio between the maximum outer diameter of the outlet portion (19) and of the longitudinal extension of the central portion (14) is from 0.5:1.0 to 2.5:1.0. Thereby, the outlet portion, forming a connection portion to the target site, may be provided as a more prominent function and the overall longitudinal dimensions of the nerve conduit may be reduced. However, it will be understood that the inner cavity of the nerve conduit may also be dimensioned for bridging a predefined gap, wherein the nerve end may be accommodated within the inner cavity at an appropriate position.

In some embodiments, the maximum outer diameter of the wall defining the outlet portion is larger than an outer diameter of the wall defining the insertion portion. Thereby, an asymmetry of the nerve conduit is provided, wherein the larger diameter of the outlet portion unambiguously indicates the portion to be connected to the target site. This also provides that the radial dimensions of the insertion portion may be less pronounced, which may be desirable both for the surrounding tissue at the implantation site and for handling during surgery.

In some embodiments, the wall of the elongate body is a single tubular wall comprising a substantially continuous thickness along the circumferential and longitudinal direction of the elongate body. Thereby, an improved structural stability and integrity may be provided for the elongate body as a whole. This may also provide similar material properties, such as a level of flexibility or rigidity, along the longitudinal extension of the elongate body and may hence also facilitate handling during implantation of the nerve conduit. In addition, the continuous thickness may facilitate manufacturing and may reduce the total amount of material required for the nerve conduit.

Moreover, the continuous wall thickness may result in a corresponding enlarged opening or cross-sectional area of the opening of the outlet portion relative to the inner cavity. The enlarged opening may furthermore improve visual feedback for the surgeon during insertion of the respective nerve end into the insertion portion and inner cavity so as to ensure that the nerve end is appropriately accommodated within the inner cavity and the terminal end is positioned at the correct longitudinal position.

In some embodiments, the inner diameter and outer diameter of the central portion are substantially continuous in the longitudinal direction of the elongate body. Accordingly, a constant inner diameter may be provided between the end portions. Thereby, accommodating and support for the nerve end may be improved, in particular, when the diameter of the inner cavity is adapted for the corresponding diameter of the nerve end to be connected to the target site. The constant inner diameter may furthermore provide an improved guiding surface for axonal growth during nerve regeneration.

As described above, in some embodiments, a cross-sectional area of the opening of the outlet portion is larger than the cross-sectional area of the inner cavity of the central portion.

In some embodiments, the cross-sectional area of the opening and/or the outer diameter of the wall of the outlet portion may increase in the longitudinal direction and away from the central portion. Such extension may result in an improved adaptability to the anatomy of the target site.

The outlet portion and the corresponding opening may be particularly formed as a rotationally symmetric shape, in some embodiments, wherein the shape is a conical shape, concave shape, funnel shape, trumpet shape, or parabolic shape. The rotational symmetry provides that the orientation of the nerve conduit during placement and connection to the target site may be facilitated. Furthermore, the mechanical properties of the outlet portion may be substantially the same along the circumference. In some embodiments, the shape of the outlet portion ensures that a gradual extension may be provided, which may further facilitate the connection to the target site, e.g. by facilitating the application of a medical adhesive. According to special embodiment, the said outlet portion and corresponding opening may be particularly formed as a rotationally symmetric shape along a longitudinal axis defined by the elongate body.

In some embodiments, a trumpet shape is used, which may comprise a substantially conical shape with an increased radial extension at the end surface. Such shape further improves the application of a medical adhesive while providing an increased contact and/or retention surface with the target site which exceeds the wall thickness of the outlet portion. The shape may also provide an optimized balance between required connection or attachment, on the one hand, and facilitating insertion into or on top of a target site, on the other hand.

In some embodiments, the wall defining the outlet portion may hence radially extend from the wall defining the central portion, at an angle between 40 degrees and less than 180 degrees, between 40 degrees and 150 degrees, between 40 degrees and 120 degrees, or between 40 degrees and 90 degrees, relative to a longitudinal axis defined by the elongate body.

An angle of between 80 degrees and 90 degrees may result in an outlet portion being substantially perpendicular to the central portion. As used herein, the expression “substantially perpendicular” includes a slight deviation from perpendicular (90°), which is illustrated in the exemplified embodiment of FIG. 6 as angle (α). In some embodiments, the angle (α) does not exceed 10°, does not exceed 7°, does not exceed 5°, or does not exceed 3°. In some embodiments, the angle (α) is in the range of 0.5°to 10°, in the range of 1° to 5°, or in the range of 1.5° to 3°. In case of a corresponding enlargement of the opening or cross-sectional area thereof, a corresponding steep inclination of the opening may be provided. In other words, a radial flare may be provided, wherein the longitudinal extension of the outlet portion is minimized.

A lower angle, e.g. between 40 and 70 degrees or 45 degrees and 65 degrees, may provide a more gradual increase of the outer diameter of the outlet portion. Accordingly, a variety of angles may be provided (including angles above 90 degrees), wherein the angle may be dependent on a predefined or required longitudinal and maximal radial extension of the outlet portion relative to the central portion or inner cavity thereof. Such longitudinal and radial extension may be dependent on the target site and surrounding tissue and, in particular, the contacting or retention surface required for appropriate connection to the target site.

In some embodiments, the wall of the outlet portion is contiguous with the wall defining the central portion and may be rounded at the interface with the wall of the central portion. In other words, the wall portions may be aligned or be in flush arrangement and are free of steps or edges at the corresponding outer surface.

According to an embodiment, the wall defining the outlet portion may be continuous in the circumferential direction. Hence, no gaps may be provided along the circumference. Such configuration may be desirable for handling purposes and to improve structural stability. Furthermore, such configuration may improve a sealing functionality of the outlet portion towards the exterior with regard to axonal growth of the nerve end accommodated in the inner cavity. By the same token, such configuration substantially avoids that surrounding tissue may impair the axonal growth and/or occurrence of neuromuscular junctions in the outlet portion.

In an alternative embodiment, the wall defining the outlet portion may be formed as a plurality of radially outward extending arms that are spaced apart from each other in the circumferential direction. Accordingly, gaps may be provided in a circumferential direction, such that the respective arms may form individual contacting surfaces with the target tissue. Thereby, an improved level of flexibility and adaptability to the anatomy of the target tissue may be provided while at the same time a medical adhesive may be applied in a manner, wherein both the amount of edges and the connecting surface of the target site covered by the medical adhesive may be increased. Furthermore, the provision of individual arms may facilitate insertion of the end surface of the outlet portion into a slit or recess of the target site, if such connection is desired for the respective treatment and/or anatomy of the target site.

In some embodiments, each of the arms has a circumferential extension of between 10 degrees and 150 degrees. Such circumferential extension may provide a sufficient structural stability and connective force while providing a desirable level of flexibility and adaptability to the target site. The circumferential extension may be adapted to the number of arms and/or predefined gap extensions.

In some embodiments, the arms are equally spaced apart from each other in the circumferential direction. This ensures that a rotational symmetry may be provided, which is desirable for correct orientation of the nerve conduit relative to the target site. Such spacing also provides that a connection to the target site may be provided in a homogeneous manner.

However, other arrangements of the arms may also be provided, wherein pairs or a multiple of adjacent arms are arranged along a respective radial axis, wherein two or more radial axes may be provided at a predefined angle to each other. Such configurations may be desirable for a corresponding extension of the anatomy of the target tissue, e.g., when the target site has a more pronounced extension in a particular direction and/or a connection to a particular portion of the target site is desirable.

Depending on the maximum outer diameter of the outlet portion and the anatomy of the target site, an appropriate number of arms may be provided to ensure proper connection to the target site and structural stability of the arms. In some embodiments, the wall is formed of 2 to 16 arms, 6 to 12 arms. For example, it may be particularly desirable in this regard if the outlet portion is formed by 10 arms, which are equally spaced apart from each other, and wherein, in some embodiments, each arm has a circumferential extension of about 20 degrees.

In order to increase the flexibility and adaptability of the respective arms, the arms may comprise a tapered portion in the radial direction. In other words, each arm may have a smaller circumferential extension, for example, at a middle portion of the arm in the radial direction, i.e. between the end of the arm connected to the central portion and an opposing free end. The tapered shape, e.g. defining an hourglass shape of the arm, may provide that the arm may be bent or deflected about the tapered section. The position of such tapered section may hence be adapted to a required surface of the end of the arm required for the connection to the target site and/or to an optional insertion depth into the target site.

To facilitate the application of a medical adhesive and adhesive strength or to increase a retention force if the outlet portion is inserted into the target site, the outlet portion may comprise a larger surface roughness at the outer surface of the wall compared with the outer surface of the wall defining the central portion. Alternatively, or in addition, such increased surface roughness may (also) be provided with regard to the outer surface of the wall defining the insertion portion. Accordingly, it may be provided that both the outlet portion and the central portion exhibit an increased surface roughness with regard to the insertion portion or that only the outlet portion exhibits such increased surface roughness. This may be dependent on the handling requirements of the nerve conduit during surgery and implantation of the nerve conduit at the target site.

In some embodiments, in order to facilitate at least partial restoration of motor control and/or restoration of sensory feedback and/or preventing of neuroma development, the outlet portion is configured for connection to muscle tissue. The muscle tissue may e.g. be a denervated target muscle, which is adjacent to or in proximity of the respective lesioned nerve end and which facilitates motor control of the injured anatomical structure, e.g. a finger. The muscle tissue may furthermore provide an effective tissue for reducing the occurrence of neuroma development, wherein a burying of a respective nerve end within the muscle may be particularly desirable.

Accordingly, the outlet portion may be configured for attachment on top of the muscle tissue or within an inner recess or slit within the muscle tissue. As described above, the outlet portion may e.g. exhibit flexibility, which may be desirable for (partial) insertion of the outlet portion into a slit of the muscle tissue and/or may provide a level of adaptability to the outer surface and corresponding anatomy of the target site. A slit of the muscle tissue may e.g. extend substantially perpendicular to a longitudinal axis defined by the elongate body, such that the outlet portion may at least partially extend into the slit. The outlet portion may hence be configured to be accommodated within the slit and be at least partially covered by the muscle tissue, such that an interference fit or positive locking may be provided. In some aspect, the target muscle tissue is used for efferent signal amplification for prosthesis control.

Alternatively, the outlet portion may be configured for connection to side of nerve. This alternative is well known in the art as end-to-side (ETS) nerve repair, in which the distal stump of a transected nerve is coapted to the side of an uninjured donor nerve. It offers a technique for repair of peripheral nerve injuries where the proximal nerve stump is unavailable or a significant nerve gap exists.

In some embodiments, while the maximum outer diameter (and/or inner diameter) of the outlet portion is larger than the maximum outer diameter of the insertion portion, the cross-sectional area of the opening of the insertion portion may also increase in the longitudinal direction and away from the inner cavity or central portion. Accordingly, the insertion portion may provide an enlarged opening to the inner cavity, which facilitates the insertion of the corresponding nerve end into the nerve conduit. As described above in view of the outlet portion, the insertion portion and opening thereof may be formed as a rotationally symmetric shape along a longitudinal axis defined by the elongate body, wherein said shape is a conical shape, concave shape, funnel shape, trumpet shape, or parabolic shape. In some embodiments, the increase in the opening is gradual and homogeneous in all radial extensions. For example, the opening may have a circular cross-section along the entire longitudinal direction of the respective end portion, wherein the diameter of the circular shape is gradually increased. Such shape furthermore has the aspect that during implantation the placement of the nerve conduit is independent from the rotational orientation. Other shapes, such as ellipsoids, may, however, also be provided, wherein the (gradual) increase in the opening is provided by an increase in at least one radial extension.

The elongate body, e.g. as a whole or the central portion and/or outlet portion and/or insertion portion, may contain a drug in its lumen or inner cavity, e.g. by coating or integrated in the material, e.g. a wall, of the respective part, which may be released over time and may e.g. facilitate nerve growth. Similarly, its lumen or inner cavity may comprise holes, pores, grooves or particular geometries or surface irregularities, and/or a filler (e.g. an hydrogel), which may facilitate the insertion, retention and/or growth of the nerve end.

In some embodiments, the outer surface of the end portions, e.g. outlet portion and/or insertion portion may also support or comprise external structures with particular geometries or surface irregularities shaped to form retention surfaces, such as holes, grooves or threads.

In particular, such external structures may desirably (i) increase the contact between the surface of the end portion and the adhesive; and/or (ii) create anchor points for the adhesive. Such external structures are disclosed, for example, in patent application PCT/EP2022/061084, the content of which is incorporated herein by reference in its entirety.

In some embodiments, the retention surfaces may be formed as at least one groove extending in a helical direction along a longitudinal axis defined by the elongate body. The helical shape may define a thread extending along the outer surface of the end portions.

In some embodiments, the wall defining the elongate body as a whole may be non-continuous in the circumferential direction. Hence, the elongate body can be opened and be used to wrap around nerve end. Such configuration may be desirable for handling purposes and to facilitate nerve insertion in the insertion portion.

According to special embodiment, the central portion 14 and inner cavity 16 are absent in the nerve conduit.

In some embodiments, the nerve conduit is formed of a biocompatible material, an inert material, a bioimplantable material, and/or a biodegradable material. The material may be chosen so as to provide a predefined structural stability while substantially avoiding or at least reducing the inflammatory response of a patient to be treated. For example, a biocompatible material may be chosen, which is gradually degrading over time after implantation yet which may initially provide sufficient structural support to adequately repair a nerve lesion and ensure that the nerve end is connected properly and with sufficient stability, e.g. during movement of the tissue.

The particular material may furthermore be chosen in order to facilitate or support nerve growth, for example, by comprising or otherwise incorporating or including a corresponding coating, e.g. with one or more biologically active agent (including neurotrophic factors). Other examples of such biologically active surface functionalities include, but are not limited to, e.g. anti-inflammatoiresuppressants, and neuroprotective agents. Biologically active agents may be surface bound and/or be entrapped in a structure defining the elongate body, e.g. the wall described in the above. Alternatively, the one or more biologically active agent is cross-linked to gel matrices within the nerve conduit of the present disclosure or directly injected into the lumen of the nerve conduit.

Examples of such biologically active agents are cytokines, nerve growth factor, hyaluronic acid, tacrolimus, cyclosporin A, melatonin, vitamin B12, methylprednisolone, riluzole, taxol, cetuximab, brain-derived neurotrophic factor (BDNF), laminin, nerve growth factor (NGF), glial cell-derived neurotrophic factor (GDNF), glial growth factor (GGF), alpha fibroblast growth factor (α-FGF), in some embodiments, an example is biologically active agents with neuroregenerative properties such as tacrolimus.

In some embodiments, the nerve conduit is formed of a polymer-based material, such as an elastomer. This has the aspect that a plurality of manufacturing methods may be applied and/or particular characteristics of the material may be provided based on e.g. a polymer unit. In particular, the polymer-based material may be a biocompatible material, which furthermore has elastic properties, such that, in the implanted states, the nerve conduit may adapt to tissue movement surrounding the repaired nerve lesion.

The nerve conduit may be formed of a polymerized and/or crosslinked polymer unit comprising an ester group component and an acid ester group component, in some embodiments, the ester group component being a polyol and the acid ester group component being a polyacid.

The material being used for the respective portions may be the same. Thereby, manufacturing may be further facilitated and structural characteristics of the nerve conduit may be substantially homogeneous along the longitudinal direction of the nerve conduit. In this matter, if the nerve conduit system is configured accordingly, biodegradation (and/or bioresorption) may also occur in a predefined or expected manner.

In some embodiments, the portions are integrally formed or formed of a single piece, e.g. by material bonding and corresponding structural integration. By providing the nerve conduit as a single piece, the robustness of the nerve conduit may be further improved since separate connections between portions are effectively avoided.

In some embodiments, the nerve conduit is formed by a 3D-printing process. This is particularly desirable when the material of the nerve conduit is polymer-based, wherein a curing of the material may be provided substantially instantaneously, for example using (UV) light. Furthermore, this provides an accuracy level that may not be (easily) achieved by extrusion and/or a dipping process. In particular, the 3-D printing process enables to obtain a particular shape of each nerve conduit, wherein, for example, the printing process may provide that particular biologically active agents are integrated in the 3-D structure according to a predefined pattern, e.g. within a mesh structure and/or in particular pockets or cavities formed by the 3-D structure. Thereby, orchestration and support of the nerve repair may be further improved and/or biodegradation may be achieved in a more controllable matter.

The above object is furthermore achieved by use of a nerve conduit described in the above for repairing, supporting, and/or guiding neural tissue, in particular for repairing a peripheral nerve lesion. Furthermore, the nerve conduit may be used in combination with a medical adhesive.

According to another aspect of the present disclosure, a method for treating a peripheral nerve lesion is suggested, comprising the steps of:
providing a nerve conduit as described in the above;
inserting a nerve end of a lesioned nerve into the insertion portion of the nerve conduit and securing said nerve end to the nerve conduit; and
connecting the nerve conduit to a target site.

In some embodiments, the securing of the lesioned nerve end is performed by applying a medical adhesive outside of the insertion portion and/or within the opening thereof via the corresponding opening. In some embodiments, the connection to the target site is furthermore performed at an outer surface of the target site or by inserting at least a portion of the outlet portion into a slit of the target site, wherein the target site is a (denervated) muscle. In some embodiments, the connection of the nerve conduit to the target site includes the application of a medical adhesive along a circumference of the outlet portion at least at the interface with the target site, e.g. target tissue.

Medical adhesive according to the present disclosure may be any medical adhesive of the art. In some embodiments, the medical adhesive is able to polymerize when exposed to light. Before such polymerization the medical adhesive may be fluid or viscous. In some embodiments, the medical adhesive is a light-curable compound. In some embodiments, said photoinitiator is sensitive to ultraviolet (UV) radiations. In some embodiments, the medical adhesive is or comprises poly glycerol sebacate acrylate (PGSA) or PGSAA (e.g., as described in WO2021/078962).

According to another aspect of the present disclosure, the method for treating a peripheral nerve lesion comprises targeted muscle reinnervation (TMR). In some embodiments, the method maximizes the number of motor axons to innerve a target muscle. In some embodiments, the method improves signal transduction from the target muscle. In some embodiments, the target muscle is used for efferent signal amplification for prosthesis control.

It is understood that the nerve conduit of the present disclosure might (also) be used for a similar application, such as for example connecting a tendon or ligament end with target bone or bone section. In this embodiment, the tendon or ligament end replaces the nerve end in all of the above description and the target site encompasses bone or bone section, and the nerve conduit is called tendon conduit or ligament conduit.

More broadly, the present disclosure thus concerns a conduit (10) for connecting a tubular structure end (26) to a target site (28), comprising an elongate body (12) formed by a wall (22) and comprising:
a central portion (14) defining an inner cavity (16) and end portions (18, 19) defining respective openings (20, 20′) to the inner cavity (16) and arranged adjacent to the central portion (14) and at longitudinally opposing ends of the elongate body (12), one end portion (18) being an insertion portion configured for inserting a respective tubular structure end (26) and the other end portion (19) being an end outlet portion, wherein an outer diameter of the wall defining the outlet portion (19) is larger than an outer diameter of the wall defining the central portion (14), and wherein the tubular structure is selected in the group consisting in nerve, tendon and ligament.

### BRIEF DESCRIPTION OF THE DRAWINGS

The present disclosure will be more readily appreciated by reference to the following detailed description when being considered in connection with the accompanying drawings in which:

FIG. 1 shows a schematic depiction of a nerve conduit according to the present disclosure in a perspective side view;

FIG. 2 schematically shows the nerve conduit according to FIG. 1 in a longitudinal section;

FIG. 3 shows a schematic depiction of a nerve conduit according to the present disclosure with an alternative outlet portion in a perspective side view;

FIG. 4 schematically shows the nerve conduit according to FIG. 3 in a side view;

FIG. 5 schematically shows the nerve conduit according to FIG. 3 in a bottom view toward the outlet portion;

FIG. 6 schematically shows the nerve conduit according to FIG. 3 in a longitudinal section;

FIG. 7 schematically depicts the nerve conduit according to the present disclosure in a connected state with a target tissue in a sectional view; and

FIG. 8 schematically depicts the nerve conduit according to the present disclosure in an alternative connected state with a target tissue in a sectional view.

### DETAILED DESCRIPTIONS OF EMBODIMENTS

In the following, the present disclosure will be explained in more detail with reference to the accompanying figures. In the Figures, like elements are denoted by identical reference numerals and repeated description thereof may be omitted in order to avoid redundancies.

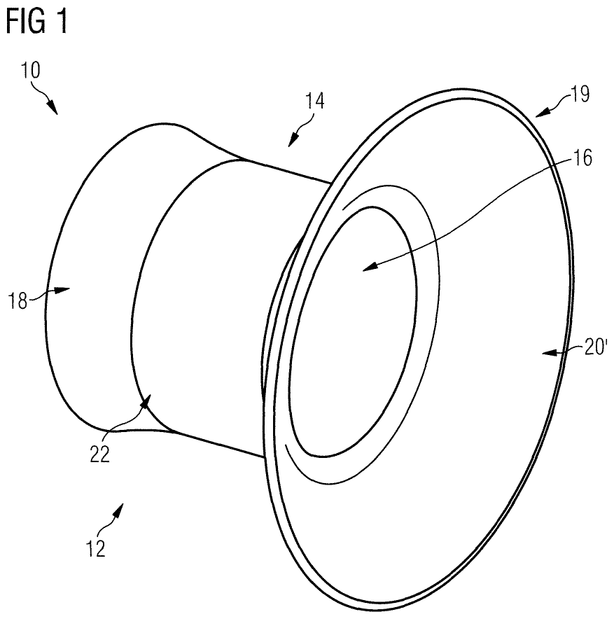

In FIG. 1 a nerve conduit 10 according to the present disclosure is schematically shown in a perspective side view. The nerve conduit 10 comprises an elongate body 12, which comprises an insertion portion 18 and an outlet portion 19 that are arranged at opposing ends of the elongate body 12 in a longitudinal direction defined by a primary extension of the elongate body 12. The insertion portion 18 is configured to receive a respective nerve end (not shown), e.g. a proximal nerve end of a lesioned nerve, which is to be connected to a target site, e.g. a target tissue, in particular a (denervated) muscle, via the outlet portion 19. Between the insertion portion 18 and the outlet portion 19, a central portion 14 is provided in a direct adjacent or contiguous manner. The central portion 14 defines an inner cavity 16, which is adapted to accommodate the nerve end received via the insertion portion 18.

Accordingly, a continuous lumen or channel of the nerve conduit 10 is provided from the opening 20 of the insertion portion 18 to the opening 20′ of the outlet portion 19. As shown, the elongate body 12 is substantially formed by a tubular wall 22 defining an inner lumen or channel of the nerve conduit 10, wherein, in some embodiments, the wall 22 comprises a substantially constant thickness (t) along the longitudinal extension of the elongate body 12.

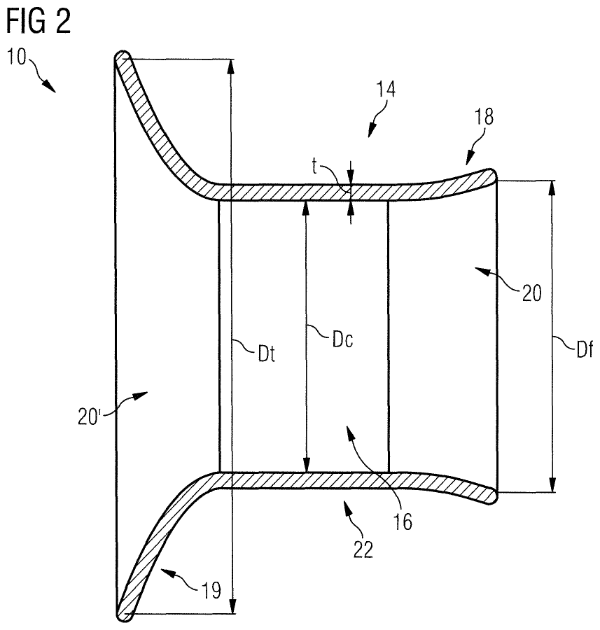

The central portion 14 and its inner cavity 16 is formed as a substantially cylindrical portion having a constant inner (Dc) (see FIG. 2) and outer diameter, which is adapted to accommodate the received nerve end having a corresponding diameter. The outlet portion 19, however, radially extends in the longitudinal direction in a direction away from the central portion 14. That is, the outer diameter and corresponding cross-sectional area of the opening 20′ gradually increase towards the longitudinal end face of the outlet portion 19. As shown, the outlet portion 19 according to the example is formed as a conical or trumpet-like portion, which is desirable for the connection to the target site, e.g. by increasing the connecting surface and/or surface for applying a medical adhesive. The conical shape furthermore may provide a form-fitting connection after curing of such medical adhesive, providing an improved retention or adhesive force at least in the longitudinal direction.

The insertion portion 18 also comprises a radially extending opening 20 and cross-sectional area thereof, which is desirable for the insertion of the nerve end into the nerve conduit and the accommodation within the inner cavity 16. The maximum radial extension (Df) of the insertion portion 18, however, does not exceed the maximum radial extension (Dt) of the outlet portion 19. Thereby, the respective functionality of the end portions may be unambiguously determined during implantation and surgery by a medical professional or surgeon.

In FIG. 2, the nerve conduit 10 according to FIG. 1 is depicted in a sectional side view. From this section it may be appreciated that the thickness (t) of the wall 22 is substantially the same for the entire elongate body 12. Furthermore, in the radial increase of the respective openings 20, 20′ of the insertion portion 18 and outlet portion 19 are more clearly depicted according to this view. Accordingly, the maximum outer diameter (Dt) of the outlet portion 19 and cross-sectional area of the corresponding opening 20′ is also shown to exceed the inner cavity 16 and outer diameter of the center portion 14 at a ratio of between about 1.5:1 to 2.5:1.

Compared with the longitudinal extension of the central portion 14, the outlet portion 19, however, is dimensioned smaller in the longitudinal direction. Thereby, a pronounced outlet portion 19 is provided, which may function as a connection portion for the target site, yet which longitudinal extension is limited to maintain the overall dimensions within a physiologically acceptable range and provide improved structural stability of the nerve conduit 10 as a whole.

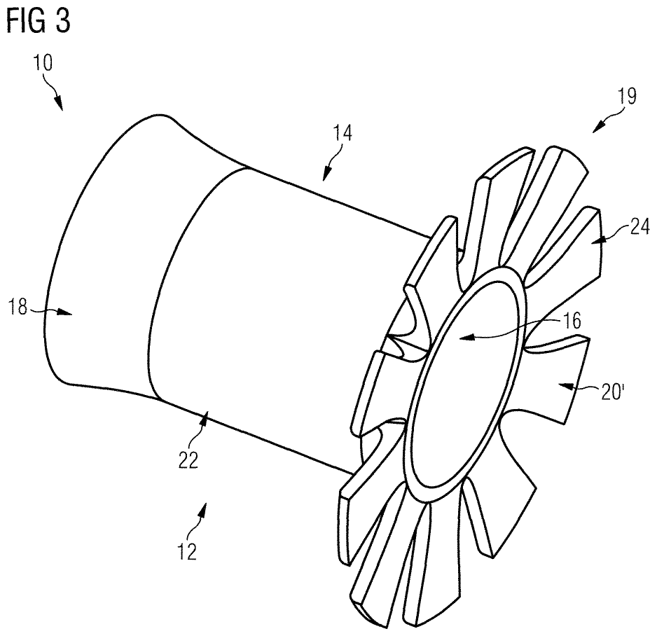

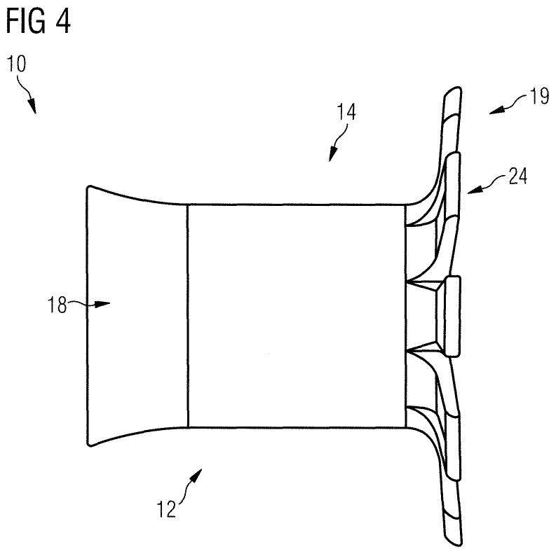

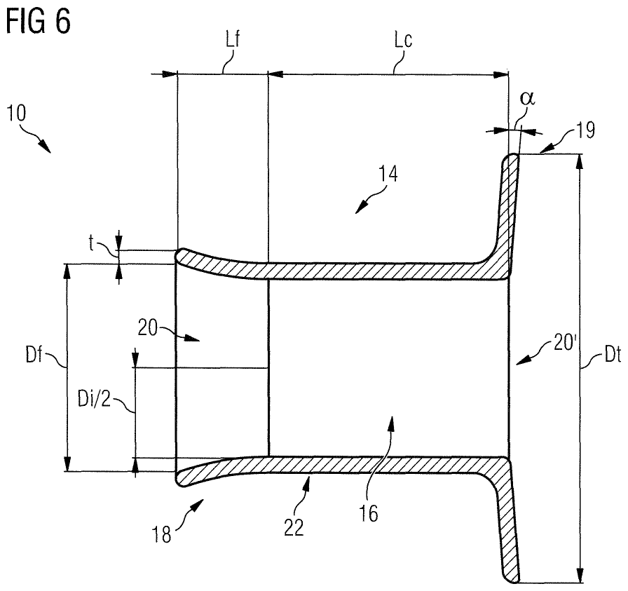

In FIGS. 3 to 6 an alternative embodiment of a nerve conduit 10 according to the present disclosure is depicted in different views. According to this embodiment, which resembles the embodiment depicted in FIGS. 1 and 2, the outlet portion 19 has been modified. Instead of a continuous surface in the circumferential direction, the wall 22 defining the outlet portion 19 is formed of a plurality of radially extending arms 24, which, in some embodiments, are equally spaced apart to each other in the circumferential direction. The arms 24 extend radially between about 80 degrees and 90 degrees with regard to the longitudinal axis at the junction with the central portion 14, such that the arms 24 form a radial flare that is substantially perpendicular to the wall of the central portion 14. This is best shown in FIGS. 4 and 6. As can be retrieved from FIG. 6, the expression “substantially perpendicular” includes a slight deviation depicted as angle (a). In some embodiments, the angle (a) does usually not exceed 10°, it does not exceed 7°, it does not exceed 5°, it does not exceed 3°. As shown, the arms 24 extend in such manner that no steps or edges are formed between the central portion 14 and the outlet portion 19 and form a rounded connecting end at the central portion 14.

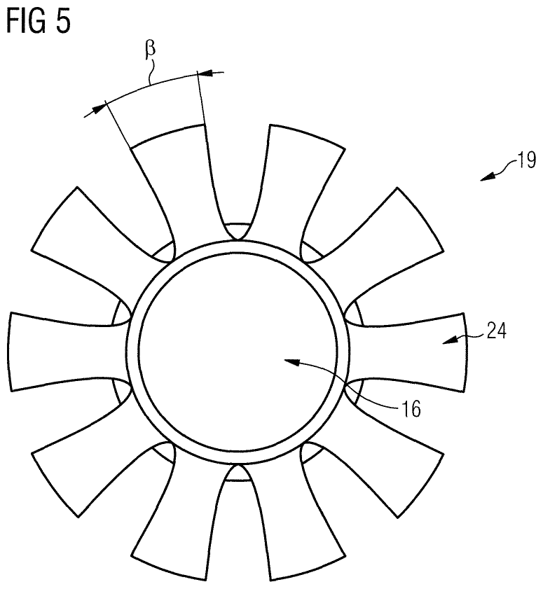

According to the present example, the outlet portion 19 is formed of 10 arms 24, which have a circumferential extension of about 20 degrees (β), as best shown in FIG. 5. Although the number of arms and the circumferential extension may vary, the exemplary configuration has been found to enable a particularly desirable flexibility and connection to the target tissue while maintaining sufficient structural stability. The flexibility of the arms 24 may furthermore be improved by an optional tapered section of the arms 24, as indicated in FIG. 5 as a middle section between the free end and the connecting end of the respective arms 24.

While the opening 20′ and cross-sectional area thereof of the outlet portion 19 also gradually increases in the embodiment according to FIGS. 1 and 2, the opening 20′ of the outlet portion 19 according to the embodiment depicted in FIGS. 3 to 6 substantially corresponds to the diameter of the inner cavity 16 of the adjacent central portion 14. Thereby, the mechanical stability of the outlet portion 19 may be improved for the respective arms 24, as indicated e.g. in FIGS. 3, 5 and 6. However, it will be understood that the opening 20′ of the outlet portion 19 may also gradually extend in the radial direction while maintaining sufficient structural stability, e.g. by increasing a thickness of the wall 22 at least at the junction between the central portion 14 and the outlet portion 19. Alternatively, it is further possible to decrease this thickness of the wall 22 to obtain flexibility of the orientation of the outlet portion 19.

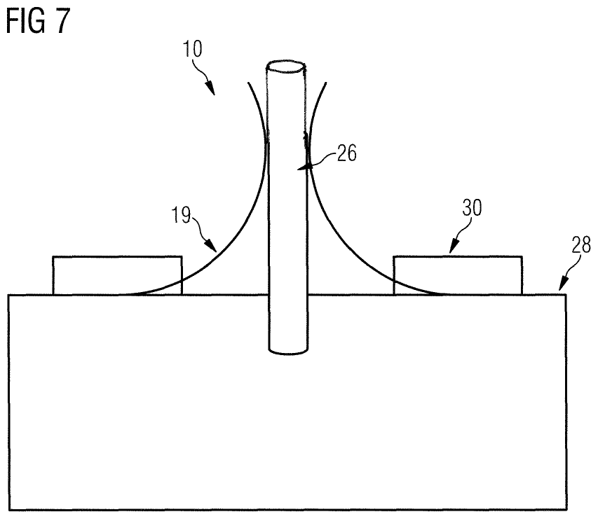

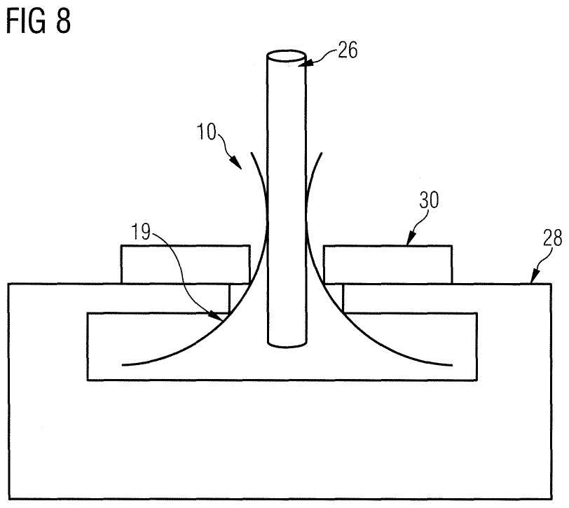

In FIGS. 7 and 8 two alternative approaches of connecting the nerve conduit 10 according to the present disclosure to a target site, such as a target tissue 28 are depicted.

According to the example shown in FIG. 7, a (proximal) nerve end 26 may be introduced into the nerve conduit 10 via the corresponding insertion portion 18 at the top end and be received by the central portion 14. The nerve end 26 may be secured to the nerve conduit 10 by a medical adhesive or by other techniques, e.g. by suturing technique. The outlet portion 19 may then be connected to the target tissue 28, e.g. a denervated muscle section, by application of a medical adhesive 30 at the interface between the outlet portion 19 and the target tissue 28. After connection and completion of the implantation, the nerve conduit 10 may facilitate axonal growth towards and into the target tissue 28, as indicated in the Figure.

In the example according to FIG. 8, the nerve end 26 may be secured to the nerve conduit 10 in the same manner. However, instead of a topical connection, i.e. at the outer surface of the target tissue 28, a slit or recess or flap of the target tissue 28 may be provided, which is configured to receive the outlet portion 19 of the nerve conduit 10, at least partially, as shown in the Figure. The flap or slit may provide an initial securing of the nerve conduit 10 to the target tissue 28. Further fixation may be provided by the application of a medical adhesive 30 to the outlet portion 19 and at the interface with the outer surface of the target tissue 28, however, in the present example, said application may be provided closer to the junction with the central portion compared with the example provided in FIG. 7. Due to the conical or trumpet-like shape of the outlet portion 19, the medical adhesive 30 may still provide a sufficient retention or adhesive force, such that the nerve conduit 10 may be securely connected to the target tissue.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the present disclosure.

### List of Reference Numerals

10 Nerve conduit
12 Elongate body
14 Central portion
16 Inner cavity
18 Insertion portion
19 Outlet portion
20, 20′ Opening
22 Wall
24 Arm
26 Nerve end
28 Target tissue
30 Medical adhesive

1. A nerve conduit (10) for connecting a nerve end (26) to a target site (28), comprising an elongate body (12) formed by a wall (22) and comprising
a central portion (14) defining an inner cavity (16) and
end portions (18, 19) defining respective openings (20, 20′) to the inner cavity (16) and arranged adjacent to the central portion (14) and at longitudinally opposing ends of the elongate body (12), one end portion (18) being an insertion portion configured for inserting a respective nerve end (26) and the other end portion (19) being a nerve end outlet portion,
wherein an outer diameter of the wall defining the outlet portion (19) is larger than an outer diameter of the wall defining the central portion (14).

2. The nerve conduit (10) according to claim 1, wherein the maximum outer diameter of the wall defining the outlet portion (19) is between 1 mm and 10 mm, preferably between 2 mm and 6 mm, larger than the outer diameter of the wall defining the central portion (14).

3. The nerve conduit (10) according to claim 1 or 2, wherein a ratio between the maximum outer diameter of the wall (22) defining the outlet portion (19) and the outer diameter of the wall (22) defining the central portion (14) is between be from 1.1:1.0 to 8.0:1.0.

4. The nerve conduit (10) according to any of the preceding claims, wherein the diameter of the inner cavity (16) is between 1 mm and 12 mm, preferably between 1.5 mm and 6.5 mm.

5. The nerve conduit (10) according to any of the preceding claims, wherein a ratio between the longitudinal extension of the outlet portion (19) and the central portion (14) is between 0.1:1.0 and 0.6:1.0.

6. The nerve conduit (10) according to any of the preceding claims, wherein a ratio between the maximum outer diameter of the outlet portion (19) and the longitudinal extension of the central portion (14) is between 1.5:1.0 and 2.5:1.0.

7. The nerve conduit (10) according to any of the preceding claims, wherein the maximum outer diameter of the wall (22) defining the outlet portion (19) is larger than an outer diameter of the wall (22) defining the insertion portion (18).

8. The nerve conduit (10) according to any of the preceding claims, wherein the wall (22) of the elongate body (12) is a single tubular wall comprising an essentially continuous thickness along the circumferential and longitudinal direction of the elongate body (12).

9. The nerve conduit (10) according to any of the preceding claims, wherein the inner diameter and outer diameter of the central portion (14) are essentially continuous in the longitudinal direction of the elongate body (12).

10. The nerve conduit (10) according to any of the preceding claims, wherein a cross-sectional area of the opening (20′) of the outlet portion (19) is larger than the cross-sectional area of the inner cavity (16) of the central portion (14).

11. The nerve conduit (10) according to any of the preceding claims, wherein the cross-sectional area of the opening (20′) and/or the outer diameter of the wall (22) of the outlet portion (19) increases in the longitudinal direction and away from the central portion (14).

12. The nerve conduit (10) according to claim 11, wherein the outlet portion (19) and the corresponding opening (20′) are formed as a rotationally symmetric shape along a longitudinal axis defined by the elongate body (12), preferably wherein said shape is selected from the group consisting of a conical shape, concave shape, funnel shape, trumpet shape, or parabolic shape.

13. The nerve conduit (10) according to claim 11 or 12, wherein the wall (22) defining the outlet portion (19) radially extends from the wall (22) defining the central portion (14) at an angle between 40 degrees and less than 180 degrees, more preferably between 40 degrees and 150 degrees, even more preferably between 40 degrees and 120 degrees, still more preferably between 40 degrees and 90 degrees relative to a longitudinal axis defined by the elongate body (12).

14. The nerve conduit (10) according to any of the preceding claims, wherein the wall (22) defining the outlet portion (19) is continuous in the circumferential direction.

15. The nerve conduit (10) according to any of claims 1 to 13, wherein the wall (22) defining the outlet portion (19) is formed as a plurality of radially outward extending arms (24) that are spaced apart from each other in the circumferential direction.

16. The nerve conduit (10) according to claim 15, wherein each of the arms (24) has a circumferential extension of from 10 degrees to 150 degrees, preferably from 10 degrees to 30 degrees.

17. The nerve conduit (10) according to claim 15 or 16, wherein the arms (24) are equally spaced apart from each other in the circumferential direction.

18. The nerve conduit (10) according to any of claims 15 to 17, wherein the wall (22) is formed of 2 to 16 arms (24), preferably 6 to 12 arms (24).

19. The nerve conduit (10) according to any of claims 15 to 18, wherein the arms (24) comprise a tapered portion in the radial direction.

20. The nerve conduit (10) according to any of the preceding claims, wherein the outlet portion (19) is configured for attachment to/connection with muscle tissue.

21. The nerve conduit (10) according to claim 20, wherein the outlet portion (19) is configured for attachment on top of the muscle tissue or within an inner recess or slit within the muscle tissue.

22. The nerve conduit (10) according to any of the preceding claims, wherein the outlet portion (19) comprises a larger surface roughness at the outer surface of the wall (22) compared with the outer surface of the wall (22) defining the central portion (14).

23. The nerve conduit (10) according to any of the preceding claims, wherein the cross-sectional area of the opening (20) of the insertion portion (18) increases in the longitudinal direction and away from the inner cavity (16).
